(19) United States
(12) Patent Application Publication
(10) Pub. No.: US 2025/0269199 A1
(43) Pub. Date: Aug. 28, 2025

(54) PHOTOTHERAPY MASK FOR FACIAL TREATMENTS WITH TARGETED ILLUMINATION AND ENHANCED COMFORT

(71) Applicant: Shenzhen Kaiyan Medical Equipment Co., Ltd., Shenzhen (CN)

(72) Inventors: Alain Dijkstra, Amstelveen (NL); LI XIANG, Shenzhen (CN); Yuan Huadong, Shenzhen (CN); Tu Mingjun, Shenzhen (CN)

(73) Assignee: Shenzhen Kaiyan Medical Equipment Co., Ltd., Shenzhen (CN)

(21) Appl. No.: 19/053,460

(22) Filed: Feb. 14, 2025

(30) Foreign Application Priority Data
Feb. 23, 2024 (CN) 2024203362272

(51) Int. Cl. A61N 5/06; A61N 1/32; A61N 5/00; H05K 7/20

(52) U.S. Cl. CPC A61N 5/0616; A61N 1/328; A61N 2005/007; A61N 2005/0647; A61N 2005/0652; A61N 2005/0654; A61N 2005/0659; A61N 2005/0663; H05K 7/2039

(57) ABSTRACT

Embodiments of the present invention provide a phototherapy mask designed to enhance user comfort and effectiveness during treatment. The mask comprises a shell that fits over the user's face, featuring a first opening to avoid contact with the eyes and a second opening to avoid the nose. A first pad, positioned around the edge of the first opening, rests against the skin near the peripheral eyes to control the distance between the mask and the skin, while a second pad, located at the bottom of the shell, supports the chin. These pads help maintain the mask's stability and prevent it from shifting during use. The light source assembly, installed on the shell, provides phototherapy to the skin.

### TECHNICAL FIELD

The present invention relates generally to phototherapy devices, specifically to a facial phototherapy mask designed to provide targeted and effective phototherapy treatments for skin rejuvenation, wrinkle reduction, and other dermatological applications.

### BACKGROUND ART

Phototherapy masks have gained popularity in recent years for their ability to provide therapeutic light treatments, particularly for skin conditions such as acne, wrinkles, and pigmentation issues. Typically, these masks consist of a flexible body made from materials such as silica gel, which is designed to conform to the contours of the face, ensuring the efficient delivery of light therapy. The mask is equipped with LED lamp beads arranged to emit specific wavelengths of light that are beneficial for skin rejuvenation or treatment.

However, the current design of phototherapy masks presents several challenges related to user comfort and the overall effectiveness of the therapy. Specifically, when the mask is secured to the face with straps, the flexible silica gel material while providing a close fit, reduces the breathability of the skin. This lack of airflow can lead to discomfort, particularly when the mask is worn for extended periods. The occlusion of airflow not only hinders comfort but may also lead to excessive sweating, irritation, or a feeling of claustrophobia, making it less ideal for prolonged usage.

Furthermore, the design of the mask, although effective in ensuring that light is directed at the skin, fails to account for the need for proper ventilation during use. Without adequate air circulation, the skin beneath the mask may become overheated or suffocated, reducing the mask's overall comfort and making it less likely for users to complete a full treatment session. This limitation underscores the need for an improved design that balances effective phototherapy with user comfort, promoting better breathability and an overall more pleasant experience during use.

Therefore, there is a need for a phototherapy mask that not only provides effective light therapy but also enhances comfort by improving breathability and reducing the discomfort associated with prolonged use.

### OBJECTS OF THE INVENTION

Some of the objects of the invention are as follows:

An object of the present invention is to provide a phototherapy mask that enhances the breathability of a user.

Another object of the present invention is to provide a phototherapy mask that provides effective skin ventilation and comfort to the user.

Another object of the present invention is to provide a phototherapy mask that can be used for extended periods without compromising skin ventilation or causing irritation, thereby providing a more comfortable and effective phototherapy experience.

Another object of the present invention is to provide a phototherapy mask with a plating area for dissipating heat generated by the LEDs.

Another object of the present invention is to provide a phototherapy mask with one or more gaskets to prevent hard contact between the mask and the skin and distance between the mask and skin is maintained.

### SUMMARY OF THE INVENTION

According to a first aspect of the present invention, a phototherapy face mask is provided. The phototherapy face mask, comprising: a shell adapted to fit on the face of a user; a first opening in the shell for eyes of the user; a second opening in the shell or nose tip of the user; a first pad arranged around the first opening, the first pad is used for resting against periphery of eyes of the user to control the distance between shell and the face of the user; a second pad installed at the bottom of the shell for supporting chin of the user; and a light source assembly in the shell for providing phototherapy to the face of the user.

In one embodiment of the invention, the light source assembly comprises a lamp layer at an inner side of the shell having a plurality of LED lamp beads arranged on the lamp layer.

In one embodiment of the invention, the plurality of LED lamp beads are red LED lamp beads and infra-red LED lamp beads.

In one embodiment of the invention, the light source assembly comprises a first illumination zone for irradiating the lips of the user to remove lip line, a second illumination zone for irradiating cheeks to lift the cheek, and a third illumination zone for irradiating chin to lift the chin.

In one embodiment of the invention, the shell comprises a first metal plating area corresponding to position of the first illumination zone, a second metal plating area corresponding to position of the second illumination zone, and a third plating area corresponding to position of the third illumination zone for dissipating heat generated by the light source assembly.

In one embodiment of the invention, the shell further comprises a light-transmitting layer and the light source assembly is positioned between the light-transmitting layer and the shell.

In one embodiment of the invention, the first pad and the second pad are made of a flexible silica gel layer.

In one embodiment of the invention, the third illumination zone covers the left side, right side and base of the chin.

In one embodiment of the invention, the shell comprises a fourth metal plating area arranged along an edge of the first opening.

In one embodiment of the invention, the shell comprises a fifth metal plating area arranged along the edge of the second opening.

In one embodiment of the invention, the phototherapy face mask further comprises a strap, the strap comprises a first band body connected to two sides of the shell and a second band body connected to top at a first end and to the first band body at a second end.

In one embodiment of the invention, the phototherapy face mask further comprises a control assembly mounted on top of the shell and a power supply assembly for driving the control assembly.

In one embodiment of the invention, the phototherapy mask further comprises one or more stimulation element.

In one embodiment of the invention, the at least one stimulation element is selected from a group consisting of but is not limited to a laser, heating element, cooling element, vibration element, electrodes, micro-current element and a combination thereof.

In one embodiment of the invention, the shell comprises a plurality of micro-current electrodes for providing micro-current therapy to the user.

In one embodiment of the invention, the first pad comprises a channel for circulating a liquid or air around periphery of eyes of the user.

In one embodiment of the invention, the liquid or air is passed through a treating element that includes but is not limited to a heating element, a cooling element or a peltier element.

According to a second aspect of the present invention, a phototherapy face mask is provided. The phototherapy face mask, comprising: a shell adapted to fit on the face of a user, the shell comprises a plurality of micro-current electrodes for providing micro-current therapy to the user; a first opening in the shell for eyes of the user; a second opening in the shell or nose tip of the user; a first pad arranged around the first opening, the first pad is used for resting against periphery of eyes of the user to control the distance between shell and the face of the user; a second pad installed at the bottom of the shell for supporting chin of the user; and wherein the first pad comprises a channel for circulating air or a liquid around periphery of eyes of the user.

In one embodiment of the invention, the phototherapy mask further comprising a light source assembly in the shell for providing phototherapy to the face of the user.

In one embodiment of the invention, the air or liquid inside the channel is treated with a treating element that includes but is not limited to a heating element, a cooling element or a peltier element.

In the context of the specification, the term “processor” refers to one or more of a microprocessor, a microcontroller, a general-purpose processor, a Field Programmable Gate Array (FPGA), or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for treatment of conditions that occur at the skin surface, such as psoriasis or infection.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first’, “second” and “third” are only used for descriptive purposes and do not implicate the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue, and white colors), Surface Mount Technology (SMT) LEDs, Bi-color LEDs, Pulse Width Modulated RGB (Red-Green-Blue) LEDs, and high-power LEDs, etc.

Materials used in one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium-doped potassium fluorosilicate in combination with a blue LED to generate red light. Additionally, near Ultraviolet (UV) LEDs may be combined with europium-based phosphors to generate red and blue lights and copper and zinc-doped zinc sulfide-based phosphors to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to Nth Degree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 shows a front view of a phototherapy mask, in accordance with an embodiment of the present invention.

FIG. 2 shows a back view of the phototherapy mask in accordance with an embodiment of the present invention.

FIG. 3 shows a cross-section of the phototherapy mask showing a structure of the inner side of the phototherapy mask, in accordance with an embodiment of the present invention.

FIG. 4 shows an exploded view of the phototherapy mask, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

In the description of the embodiments of the present application, it is to be understood that the orientations or positional relationships indicated by the terms “length”, “width”, “upper”, “down”, “front”, “rear”, “left”, “right”, “vertical”, “horizontal”, “top”, “bottom”, “inner”, “outer”, etc., are based on the orientations or positional relationships shown in the drawings and are only for the convenience of describing the embodiments of the present application and for simplifying the description, and are not intended to indicate or imply that the device or element referred to must have a particular orientation, be constructed and operated in a particular orientation, Therefore, it cannot be construed as a limitation on the present application.

In addition, the terms “first” and “second” are used for descriptive purposes only and should not be construed as indicating or implying relative importance or implying the number of technical features indicated. Thus, defining the “first” and “second” features may explicitly or implicitly include one or more of these features. In the description of the embodiments of the present application, “plurality” means two or more of them, unless otherwise expressly and specifically qualified.

In the embodiments of the present application, unless otherwise expressly specified and limited, the terms “mounted”, “connected”, “connected”, “fixed”, etc., shall be construed broadly, for example, they may be fixed, detachable, or integral; It can be mechanically or electrically connected; It can be directly connected or indirectly connected through an intermediate medium, it can be the internal connection of two elements or the interaction relationship between two elements. For those of ordinary skill in the art, the specific meaning of the above terms in the embodiments of the present application may be understood according to the specific circumstances.

Embodiments of the present invention disclose a phototherapy mask designed for facial treatments, incorporating several features to enhance comfort and effectiveness. The phototherapy mask comprises a shell for adapting to the face of a user. The bottom of the shell is extended longitudinally inwards to cover the base of the chin. The phototherapy mask is designed to cover the face of the user and the base of the chin so that the phototherapy face mask is securely fixed on the face of the user.

The shell has a first opening to avoid the eyes of the user and a second opening at the tip of the nose of the user. The edge of the first opening is provided with a first pad for resting against the peripheral skin of the eyes to control the distance between the shell and the skin. The phototherapy mask further comprises a second pad installed at the bottom of the shell. The shell is provided with a light source assembly for providing phototherapy to the user's skin. The irradiation of the light source is divided into one or more illumination zones, such as a first illumination zone for irradiating the lip to remove lip lines, a second illumination zone for irradiating the cheeks, and a third illumination zone for irradiating the chin area.

The first illumination zone surrounds the lip are of the user. The second illumination zone corresponds to the position around the cheeks area. The third illumination zone is present at the bottom of the phototherapy mask to cover the chin area. The third illumination zone spread from center of the bottom of mask towards left side and ride side of chin and extended to cover the base of the chin. The bottom portion of the mask is extended longitudinally inwards to cover the base of the chin.

The outer side of the shell of the phototherapy mask is further provided with a first plating area corresponding to the first illumination zone, a second plating area corresponding to the second illumination zone, and a third plating area corresponding to the position of the third illumination zone, a fourth plating area along the edge of the first opening, and a fifth plating area corresponding to the second opening.

The first pad is provided with a channel for circulating air or liquid around the eyes of the user. The air or liquid is circulated by a pump or a motor provided in the mask. The air or liquid is passed through a heating element, a Peltier element, or a cooling element to treat the air for enhancing therapeutic effects around the eyes.

The shell comprises a plurality of micro-current electrodes arranged along the phototherapy mask. The plurality of micro-current electrodes are used for providing micro-current therapy to the face of the user.

Several additional functionalities may also be added to the phototherapy mask. For example, the phototherapy mask may comprise a power source and a control assembly mounted on the top of the shell. Moreover, the control assembly is connected to an external device to control the functioning of the phototherapy mask.

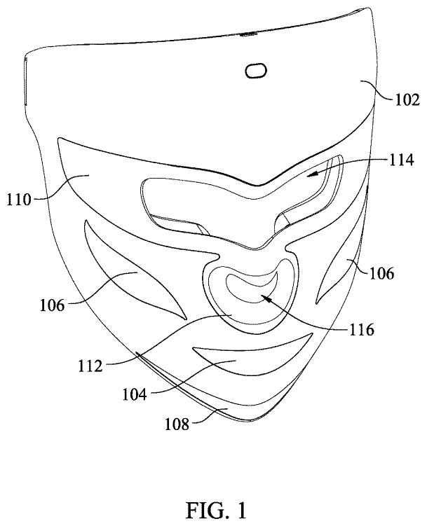

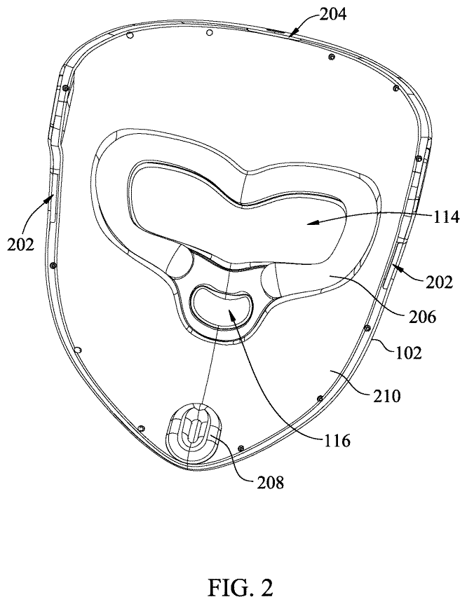

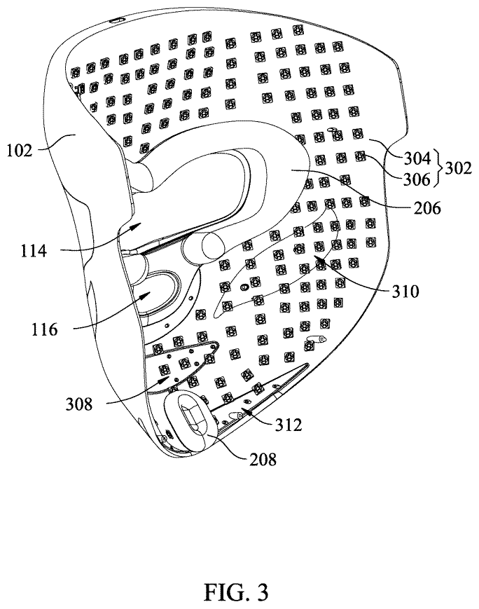

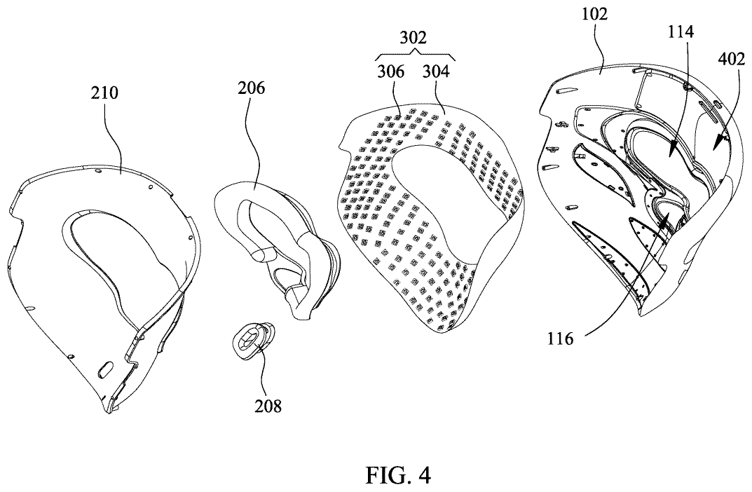

Several embodiments of the present invention will now be described in detail with references to FIGS. 1 to 4.

Referring to FIG. 1 to FIG. 4, a phototherapy mask for providing effective and comfortable phototherapy treatment to the face is provided. The phototherapy mask comprises a shell 102, a first pad 206, a second pad 208, and a light source assembly 302. The shell 102 is used for adapting with the user's face, the first opening 114 and the second opening 116 are formed in the shell 102. The first opening 114 is used to avoid the user's eyes, and the second opening 116 is used to avoid the tip of the user's nose. The first pad 206 is used for leaning against the skin of the peripheral eye to control the distance between the shell 102 and the skin. The first pad 206 is arranged around the edge of the first opening 114. The second pad 208 is used for leaning against the user's chin to maintain the stability of the distance between the shell 102 and the chin, and the second pad 208 is installed at the bottom of the shell 102. The light source assembly 302 is used for performing phototherapy on the user's skin, and the light source assembly 302 is installed on the shell 102. The first opening 114 helps the user's eyes to avoid the shell 102 thereby preventing blocking the user's vision when the user uses the phototherapy mask. The second opening 116 can avoid the user's nose tip, to avoid affecting the user's breathing. By positioning the first pad 206 to support the skin around the eye periphery and the second pad 208 against the chin, the shell 102, due to its structural strength, maintains a certain distance from the skin on the face. This design ensures the shell 102 remains stable, preventing it from swinging back and forth. Additionally, by keeping the shell 102 from directly contacting the skin, breathability, and comfort are preserved, enhancing the overall user experience. Furthermore, the first pad 206 and second pad 208 help increase the distance between the light source assembly 302 and the skin. This adjustment improves the illumination range and enhances uniformity. When the light angle of the phototherapy source is limited, a closer distance can result in a smaller illumination range, which is avoided by this design.

In an embodiment of the present application, referring to FIG. 1 to FIG. 4, the light source assembly 302 comprises a lamp layer 304 adapted to the inner side of the shell 102 and a plurality of LED lamp beads 306 are arranged on the lamp layer 304. The plurality of LED lamp beads 306 are dispersed on the lamp layer 304. The plurality of LED lamp beads 306 enhances the light intensity and uniformity and is conducive to reducing the light angle. The plurality of LED lamp beads 306 can be arranged at equal intervals, or maintain a certain spacing range so that the light distribution can be made relatively uniform.

The LED lamp beads 306 may include at least one of the white LED lamp beads, purple LED lamp beads, blue LED lamp beads, water blue LED lamp beads, green LED lamp beads, yellow LED lamp beads, or red LED lamp beads so that at least one phototherapy effect can be realized.

Referring to FIG. 1 to FIG. 4, the light source assembly 302 comprises a first illumination zone 308 for irradiating the lip to remove lip lines, a second illumination zone 310 for irradiating the cheeks to lift the cheeks, and a third illumination zone 312 for irradiating the chin to lift the chin. The three light zones can be used to achieve separate phototherapy effects on the three areas of the face, to achieve local phototherapy, and help to save energy and improve the beauty effect of the face.

In an embodiment of the present application, a red LED lamp bead and an infrared LED lamp bead are arranged in the first illumination zone 308. The red LED lamps and infrared LED lamps are effective in removing lip lines.

In an embodiment of the present application, the second illumination zone 310 is provided with a red LED lamp bead and an infrared LED lamp bead. The red LED and infrared LED lamps are used to lift the skin on the cheeks.

In an embodiment of the present application, the third illumination zone 312 is provided with a red LED lamp bead and an infrared LED lamp bead. The red LED and infrared LED lamp beads are used to lift the chin skin.

In an embodiment of the present invention, led lamp beads of the first illumination zone 308, the second illumination zone 310, and the third illumination zone 312 are can act is a similar manner or can be controlled separately using a control assembly. The first illumination zone 308, the second illumination 310, and the third illumination zone 312 can be regulated and controlled separately to provide different phototherapy effect.

In an embodiment of the present application, the outer side of the shell 102 is provided with a first metal plating area 104, a second metal plating area 106, and a third metal plating area 108. The first metal plating area 104 is located at a position on the shell 102 facing away from the first illumination zone 308. The second metal plating area 106 is located at a position on the shell 102 that is facing away from the second illumination zone 310. The third metal plating area 108 is positioned at a position on the shell 102 facing away from the third illuminating zone 312. The first metal plating area 104, the second metal plating area 106, and the third metal plating area 108 improve the aesthetic of the device and facilitate the heat dissipation of the three light zones.

In one embodiment of the present application, the phototherapy mask further comprises a translucent layer 210. The translucent layer 210 is covered on the shell 102, and the light source assembly 302 is positioned between the translucent layer 210 and the shell 102. The translucent layer 210 protects the light source assembly 302 and the LED lamp beads 306 are prevented from being exposed.

In one embodiment of the present application, the first pad 206 and/or the second pad 208 are flexible silicone parts. This reduces pressure on the skin and makes the phototherapy mask more comfortable for the user.

In one embodiment of the present application, the shell 102 is a plastic part. The use of plastic parts makes the phototherapy mask light-weight, and easy to process, thereby conducive to reducing costs.

In one embodiment of the present application, the translucent layer 210 is made of a silica gel layer. The silicone layer has good anti-aging performance, which is conducive to improving the shelf-life. The silicon layer being a softer material avoids scratching the skin during wearing.

In one embodiment of the present application, the height of the first pad 206 and/or the second pad 208 protruding from the translucent layer 210 is 1 mm-30 mm. Alternatively, the height of the first pad 206 protruding from the translucent layer 210 is 5 mm, 10 mm, 15 mm, or 20 mm, etc., and the height of the second pad protruding from the translucent layer 210 is 5 mm, 10 mm, 15 mm or 20 mm, etc. The different heights are used to control the spacing between the translucent layer 210 and the skin.

In one embodiment of the present application, the shell 102 is provided with a fourth metal plating area 110, and the fourth metal plating area 110 is arranged on the outer side of the shell 102 along the edge of the first opening 114. The use of a fourth metal plating area 110 improves the appearance quality of the phototherapy mask and facilitates heat dissipation.

In one embodiment of the present application, the shell 102 is provided with a fifth metal plating area 112, and the fifth metal plating area 112 is arranged on the outside of the shell 102 along the edge of the second opening 116. The use of a fifth metal plating area 112 improves the appearance quality of the phototherapy mask and facilitates heat dissipation.

In one embodiment of the present application, the phototherapy mask also comprises a strap (not shown) that comprises a first band body connected to both sides of the shell 102 along the width direction and a second band body connected to the top of the shell 102, and the other end of the second band body is connected to the first band body. The strap is attached to the shell 102, allowing for easy wearing of the phototherapy mask. The first band body helps prevent the shell 102 from moving left and right on the user's face, while the second band body, in conjunction with the second pad 208, restrains the shell 102 from swinging up and down. Optionally, the sides of the shell 102 are equipped with a first connecting point 202, and the top of the shell features a second connecting point 204. The first connecting point 202 is linked to the first band body, and the second connecting point 204 is linked to the second band body. This setup ensures that the shell 102 is securely connected to both the first and second band bodies.

In one embodiment of the present application, the phototherapy mask further comprises a control assembly mounted on the top of the shell 102 and a power supply assembly for driving the control assembly. The control assembly is electrically connected with the power supply assembly and the light source assembly 302. The control component can be a structure such as a circuit board, and the power component can be a rechargeable battery. The control assembly and the power supply assembly are used to control the light source assembly 302 for carrying out phototherapy. The control assembly 302 controls the led lamp beads in the first illumination zone, the second illumination zone and the third illumination zone. The control assembly and power supply assembly are mounted on the top of the shell 102, enhancing the mask's symmetry and providing a relatively flat surface for ease of processing and assembly. Optionally, the top of the shell 102 features a mounting position 402, where both the control assembly and power supply assembly are securely installed. This mounting position can take the form of a slot or similar feature, ensuring stable placement of the components. Additionally, the control assembly can wirelessly connect to devices such as a mobile phone or computer, allowing for convenient control of the phototherapy mask during use.

In an embodiment, the first pad is provided with a channel for circulating air or liquid around the eyes of the user. The air or liquid is circulated by a pump or a motor provided in the mask. The air is passed through a treating element to condition the air or liquid circulating inside the channel. The treating element includes but is not limited to a heating element, a Peltier element, or a cooling element to treat the air for enhancing therapeutic effects around the eyes. The phototherapy mask comprises a controller for controlling the pump, heating element, cooling element, and the Peltier element. The pump, heating element, cooling element, and the Peltier element are connected to the power supply assembly.

In an embodiment of the present invention, the shell comprises a plurality of micro-current electrodes arranged along the phototherapy mask. The plurality of micro-current electrodes are used for providing micro-current therapy to the face of the user. The micro-current electrode is connected to the power source assembly.

In an embodiment of the present invention, the device is configured to connect with an external communication device over a communication interface. The control assembly includes a processor, a memory unit, and a communication interface. The communication with the external communication device is routed through the communication interface. The control assembly is configured to connect with the external communication device and receives an input. The input may correspond to activating the light source assembly 302 for phototherapy or controlling one or more parameters of operating the light source assembly 302. The external communication device may be a smartphone, a desktop computer, a tablet, a Personal Digital Assistant (PDA), a smartwatch, or the like.

In an embodiment of the present invention, the phototherapy mask comprises at least one stimulation element to provide additional therapy to the user. The at least one stimulation element is selected from a group consisting of lasers, heating elements, cooling elements, vibration elements, electrodes, micro-current elements, and a combination thereof. The micro-current element provides micro-current therapy to the user, the vibration element provides massage therapy and the heating element provides heat therapy.

In an embodiment of the present invention, the phototherapy mask further comprises one or more sensors to sense one or more parameters of the skin. The phototherapy mask may communicate one or more parameters of the skin to the external communication device. The phototherapy mask on sensing the one or more parameters of the skin may regulate the operation of one or more stimulation elements.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A phototherapy face mask, comprising:
a shell adapted to fit on the face of a user;
a first opening in the shell for eyes of the user;
a second opening in the shell or nose tip of the user;
a first pad arranged around the first opening, the first pad is used for resting against periphery of eyes of the user to control the distance between shell and the face of the user;
a second pad installed at the bottom of the shell for supporting chin of the user; and
a light source assembly in the shell for providing phototherapy to the face of the user.

2. The phototherapy face mask of claim 1, wherein the light source assembly comprises a lamp layer at an inner side of the shell having a plurality of LED lamp beads arranged on the lamp layer.

3. The phototherapy face mask of claim 2, wherein the plurality of LED lamp beads are red LED lamp beads and infra-red LED lamp beads.

4. The phototherapy face mask of claim 1, wherein the light source assembly comprises a first illumination zone for irradiating the lips of the user to remove lip line, a second illumination zone for irradiating cheeks to lift the cheek, and a third illumination zone for irradiating chin to lift the chin.

5. The phototherapy face mask of claim 4, wherein the shell comprises a first metal plating area corresponding to position of the first illumination zone, a second metal plating area corresponding to position of the second illumination zone, and a third plating area corresponding to position of the third illumination zone for dissipating heat generated by the light source assembly.

6. The phototherapy face mask of claim 1, wherein the shell further comprises a light-transmitting layer and the light source assembly is positioned between the light-transmitting layer and the shell.

7. The phototherapy face mask of claim 1, wherein the first pad and the second pad are made of a flexible silica gel layer.

8. The phototherapy face mask of claim 5, wherein the third illumination zone covers the left side, right side and base of the chin.

9. The phototherapy face mask of claim 1, wherein the shell comprises a fourth metal plating area arranged along an edge of the first opening.

10. The phototherapy face mask of claim 1, wherein the shell comprises a fifth metal plating area arranged along the edge of the second opening.

11. The phototherapy face mask of claim 1, wherein the phototherapy face mask further comprises a strap, the strap comprises a first band body connected to two sides of the shell and a second band body connected to top at a first end and to the first band body at a second end.

12. The phototherapy face mask of claim 1, wherein the phototherapy face mask further comprises a control assembly mounted on top of the shell and a power supply assembly for driving the control assembly.

13. The phototherapy face mask of claim 1, wherein the phototherapy mask further comprises one or more stimulation element.

14. The phototherapy face mask of claim 13, wherein the at least one stimulation element is selected from a group consisting of but is not limited to a laser, heating element, cooling element, vibration element, electrodes, micro-current element and a combination thereof.

15. The phototherapy face mask of claim 1, wherein the shell comprises a plurality of micro-current electrodes for providing micro-current therapy to the user.

16. The phototherapy face mask of claim 1, wherein the first pad comprises a channel for circulating a liquid or air around periphery of eyes of the user.

17. The phototherapy face mask of claim 16, wherein the liquid or air is passed through a treating element that includes but is not limited to a heating element, a cooling element or a peltier element.

18. A phototherapy face mask, comprising:
a shell adapted to fit on the face of a user, the shell comprises a plurality of micro-current electrodes for providing micro-current therapy to the user;
a first opening in the shell for eyes of the user;
a second opening in the shell or nose tip of the user;
a first pad arranged around the first opening, the first pad is used for resting against periphery of eyes of the user to control the distance between shell and the face of the user;
a second pad installed at the bottom of the shell for supporting chin of the user; and
wherein the first pad comprises a channel for circulating air or a liquid around periphery of eyes of the user.

19. The phototherapy face mask of claim 18 further comprising a light source assembly in the shell for providing phototherapy to the face of the user.

20. The phototherapy face mask of claim 18, wherein the air or liquid inside the channel is treated with a treating element that includes but is not limited to a heating element, a cooling element or a peltier element.
